(19) United States
(12) Patent Application Publication
(10) Pub. No.: US 2026/0213009 A1
(43) Pub. Date: Jul. 23, 2026

(54) Systems And Devices For Collecting Health Data And Methods Of Using Same

(71) Applicant: Hill's Pet Nutrition, Inc., Overland Park, KS (US)

(72) Inventors: Susan Marie WERNIMONT, Lawrence, KS (US); John Michael MCCOY, JR., Topeka, KS (US); Austin REED, Topeka, KS (US); Cheryl STIERS, Topeka, KS (US); Mark NEAVITT, Oskaloosa, KS (US); Robin THOMPSON, Ryton (GB); Wendy Jean STEPHENS-RAWLEY, Oskaloosa, KS (US); Michael Edward MARINELLO, Highland Village, TX (US)

(73) Assignee: Hill's Pet Nutrition, Inc., Overland Park, KS (US)

(21) Appl. No.: 19/137,108

(22) PCT Filed: Dec. 13, 2023
(86) PCT No.: PCT/US2023/083791

§ 371 Date: Jun. 9, 2025

Related U.S. Application Data
(60) Provisional application No. 63/432,487, filed on Dec. 14, 2022.

(51) Int. Cl. G16H 40/67; G16H 10/60; G16H 20/30; G16H 50/30

(52) U.S. Cl. CPC G16H 40/67; G16H 10/60; G16H 20/30; G16H 50/30

(57) ABSTRACT

Technologies are disclosed for assessing a subject's health based on health-related information. Devices and/or techniques may include a memory, a display device, a processor, and/or a transceiver. The transceiver may be in communication with one or more sensors. The one or more sensors may be configured to capture one or more characteristics corresponding to the subject. The processor may be configured to receive one or more characteristics corresponding to the subject via the transceiver. The processor may be configured to determine at least a first indication of the subject's health based, at least in part, on the one or more characteristics corresponding to the subject. The processor may be configured to provide a visual representation of the first indication of the subject's health via the display device.

### CROSS-REFERENCE TO RELATED APPLICATIONS

This application claims the benefit of priority from U.S. Provisional Application No. 63/432,487, filed Dec. 14, 2022, the contents of which are hereby incorporated herein by reference in their entirety.

### BACKGROUND

People and animals/pets (e.g., subjects) can exhibit signs and/or symptoms of either major or minor health issues/conditions. Subjects might exhibit/demonstrate signs/symptoms such as fever, chills, cough, shortness of breath or difficulty breathing, fatigue, muscle or body aches, headache, sore throat, congestion, runny nose, nausea or vomiting, diarrhea, incontinence, and/or weight loss/gain, among others.

Subjects that are animals might exhibit/demonstrate signs/symptoms more particular to animals, such as barking, hissing, growling, purring, whining/whimpering, among others.

Sometimes, subjects may exhibit/demonstrate the signs/symptoms in dramatic and/or frequent ways/occasions. Sometimes, subjects may exhibit/demonstrate the signs/symptoms in infrequent, isolated, and/or subtle ways.

### SUMMARY

The disclosure comprises an electronic data collection instrument, which is more than an interactive bed (e.g., a reposing surface) or a sleep aid or a calming/soothing device. The purpose of this bed is to collect health data on the subject using embedded sensors and data capture instruments. This invention may be washable, chew and claw resistant, moisture (vomit, hairball, urination, defecation, drool/saliva, blood, mucus) resistant, comfortable, and some versions may be easily moved or portable. Some functionality of the smart bed can be used with or without a commercial bed. Features can be turned off and on to provide a tailored solution for a given situation. One or more scenarios may comprise one or more load cells, an RTD for temperature, and/or a RFID for subject identification and/or respiratory rate to collect sleep quality metrics from a specific subject.

In one or more scenarios, combinations of physiological measurements may be used for identification, which may include weight, temperature, heart rate, respiration rate, patterns of movement, time of day, frequency, etc. In one or more scenarios, identification could involve the use of a wearable device or other collar worn or implanted/injected device to interact with the bed to identify the subject (e.g., pet). In one or more scenarios, identifying the subject (e.g., pet) could rely on audio signals or computer vision/images corresponding to the subject (e.g., pet).

One or more algorithms may alert the personnel associated with the subject and/or subject if their pet or the subject is gaining or losing weight over a specific period of time. The bed can integrate with an RFID scanner, and/or other identification techniques described herein, to assist in the identification of the animal being weighed in a clinical setting, eliminating the need to transcribe weights. The bed can include a Bluetooth Low Energy (BLE) beacon to alert the personnel associated with the subject and/or subject when the pet and/or subject is in the bed, or how often the pet and/or subject visits the bed.

Audible collection can be incorporated into the bed to capture snoring, breathing issues, barking, whining, whimpering, chewing, purring, meowing, growling, vomiting, hairballs, urination, defecation, scratching, digging, seizures, hissing, respiratory rate, restlessness and other noises. Audible collection could also capture snoring, sleep apnea, asthma, other breathing issues (e.g., wheezing, and/or coughing, etc.), barking, hissing, tearing, GI function(s) (e.g., burping, gas, bowel sounds), breathing sounds/respiration (e.g., respiration rate, tachypnea, bradypnea, sleep apnea, changes in respiration rate over time, max/min/mean respiration rate, etc.), and/or disturbed sleep (e.g., movement, tossing/turning, walking/pacing, restlessness, etc.). A camera can be installed adjacent to the bed to capture images of the subject and/or movements that are captured by the load cells and integrated motion sensors.

Heating or cooling of the bed could provide a comfortable environment for the subject (e.g., human, dog/pet, etc.). The bed/reposing surface could detect the presence of vomit, hairballs, urine, blood, mucus, drool/saliva, or feces left on the bed after the subject leaves to alert the personnel associated with the subject and/or subject to the situation. Using the audio capture along with motion sensors and an integrated external camera, the bed could alert the subject and/or those responsible for the subject to a possible seizure event occurring based on one or more algorithms.

Technologies are disclosed for assessing a subject's health based on health-related information. Devices and/or techniques may include a memory, a display device, a processor, a camera, an audio recording device, and/or a transceiver. The transceiver may be in communication with one or more sensors. The one or more sensors may be configured to capture one or more characteristics corresponding to the subject. The processor may be configured to receive one or more characteristics corresponding to the subject via the transceiver. The processor may be configured to determine at least a first indication of the subject's health based, at least in part, on the one or more characteristics corresponding to the subject. The processor may be configured to provide a visual representation of the first indication of the subject's health via the display device.

Technologies are disclosed for one or more methods and/or techniques for assessing a subject's health based on health-related information. One or more techniques may comprise communicating, for example via a transceiver, with one or more sensors. The one or more sensors may be configured for capturing one or more characteristics corresponding to the subject. One or more techniques may comprise receiving, for example via the transceiver, one or more characteristics corresponding to the subject. One or more techniques may comprise determining, for example via a processor, at least a first indication of the subject's health based, at least in part, on the one or more characteristics corresponding to the subject. One or more techniques may comprise providing, for example via a display device, a visual representation of the first indication of the subject's health.

In one or more scenarios, the at least first indication of the subject's health may be substantially similar, if not identical, to the one or more characteristics corresponding to the subject.

### BRIEF DESCRIPTION OF DRAWINGS

The elements and other features, advantages and disclosures contained herein, and the manner of attaining them, will become apparent and the present disclosure will be better understood by reference to the following description of various examples of the present disclosure taken in conjunction with the accompanying drawings, wherein:

FIG. 1 illustrates an example diagram of a load cell as installed with/integrated with a reposing surface.

FIG. 2 illustrates a closer view of the load cell of FIG. 1

FIG. 3 is an example diagram of an example process for assessing a subject's health based on health-related information.

FIG. 4 is a block diagram of a hardware configuration of an example device that may function as a process control device/logic controller for the smart bed/reposing surface.

FIG. 5 illustrates a perspective view of an example of a smart bed/reposing surface base.

FIG. 6 illustrates a perspective view of smart bed/reposing surface where a bed/resting frame is separated from the reposing surface.

FIG. 7 illustrates a perspective view of an example of the reposing surface in isolation.

FIG. 8 illustrates an example of a perspective view of an example of a smart bed/reposing surface base.

FIG. 9 illustrates an example of a perspective view of an example of the bottom side of the reposing surface.

FIG. 10 illustrates an exploded view of an example of a smart bed/reposing surface base.

FIG. 11 is an example illustration of the electronics assembly of FIG. 10.

FIG. 12 is an example illustration of the electronic junction box/summing card assembly of FIG. 10.

FIG. 13 is an example illustration of how electronics and/or control devices associated with the smart bed/reposing surface may be powered for operation and/or communication.

FIG. 14 is an example illustration of how the electronic junction box/summing card assembly of FIG. 12 may be placed into electronic communication with the electronics assembly of FIG. 11.

FIG. 15 is an example of how a first load cell of FIG. 9 may be placed into electronic communication with the electronics assembly of FIG. 11 via the electronic junction box/summing card assembly of FIG. 12.

FIG. 16 is an example of how a second load cell of FIG. 9 may be placed into electronic communication with the electronics assembly of FIG. 11 via the electronic junction box/summing card assembly of FIG. 12.

FIG. 17 is an example of how a third load cell of FIG. 9 may be placed into electronic communication with the electronics assembly of FIG. 11 via the electronic junction box/summing card assembly of FIG. 12.

FIG. 18 is an example of how a fourth load cell of FIG. 9 may be placed into electronic communication with the electronics assembly of FIG. 11 via the electronic junction box/summing card assembly of FIG. 12.

FIG. 19 is an example diagram of a health parameter collection network that includes a smart bed.

### DETAILED DESCRIPTION

For the purposes of promoting an understanding of the principles of the present disclosure, reference will now be made to the examples illustrated in the drawings, and specific language will be used to describe the same. It will nevertheless be understood that no limitation of the scope of this disclosure is thereby intended.

The Smart Bed/Smart Pet Bed/Reposing Surface is a smart device designed to gather data on the health and well-being of the subject (e.g., human, animal, pet, etc.). The features of the device that make it uniquely suited to use (e.g., in a clinical and/or in-home environment, a group-housed environment, which could include a pet shelter, etc.) include comfort, ease of cleaning (e.g., washdown rated), chew/scratch resistance, dig/claw resistance, moisture resistance, repeatability and accuracy of measurements, and wired or wireless data collection and transmission (e.g., PLC, Wi-Fi, and/or Bluetooth Low Energy (BLE) based communications, among others). The device is different from that of a standard bed, as it is intended as a data collection instrument incorporating sensors for detection of health endpoints related to physical and/or cognitive and/or social/emotional health. Detection/assessment can be made of signs of conditions such as weight gain, restlessness, breathing difficulty, temperature changes and/or illness, etc. The bed could be used for early identification of the initial signs of a condition, and/or it could also be used for monitoring the response to nutrition or medical or pharmaceutical interventions.

The bed/reposing surface may incorporate or be paired with environmental sensors such as proximity beacons, audio sensors, video sensors, RFID sensors, etc., to create a data collection ecosystem to capture additional health metrics. The data collected by the bed and the associated data collection ecosystem may be analyzed/processed using one or more algorithms developed for interpretation of the health-related data. Some data processing may occur on the device and other processing may occur remotely such as on a server or in the cloud. Data processing could also occur on a mobile device paired with the reposing surface/smart bed such as a cell phone or tablet, and/or it could occur on an edge computing device such as a router.

Different iterations of the bed may incorporate additional features tailored to the needs of a particular population, for example special form factor/design or data collection features to provide comfort and collect data on aging subjects, critically ill subjects, specific types of subjects (e.g., features specific for cats or dogs, some people), young subjects (e.g., children, puppies/kittens), and more.

The smart pet bed/reposing surface may provide an overall solution to the problem of subjects and/or personnel associated with subjects (e.g., pet owners, parents of children, guardians, doctors, veterinarians, etc.) having a difficult time assessing the health of the subject. For example, obesity is a common problem among both companion animals (cats and dogs) and humans (e.g., subjects). Of approximately 90% of subjects (e.g., pets/humans) that may be overweight or obese, such subjects or those responsible for such subjects may be unaware of the overweight or obese condition. With this smart bed/reposing surface, a standard pet owner and/or subject would have the opportunity to not only measure and quantify the subject's weight objectively, but also see how the subject's weight is changing over time. Such weight information may be statistically compared to an average, median, or other weight metric, perhaps for example based on the subject's age, race, breed, life stage, medical history (e.g., other subjects with kidney disease, arthritis, etc.), nutritional intake (e.g., subjects receiving various nutrition regimens, etc.).

Weight is a health metric important to both pet owners and/or subjects and clinicians that the disclosed device can quantify, however, the device is proposed to quantify other important health metrics, including temperature, sleep quality, breathing rate, heart rate, movement patterns, social interaction among subjects, etc. For example, early detection of a fever and/or high temperature could be crucial in terms of the detection of an infection. For dogs and cats specifically, the standard temperature measurement that is considered most accurate is a rectal temperature. The device may also capture and/or be paired with devices that capture audio or visual signals such as audio recordings or visual images or video recordings. The device could also be paired with streaming video instances such as telemedicine.

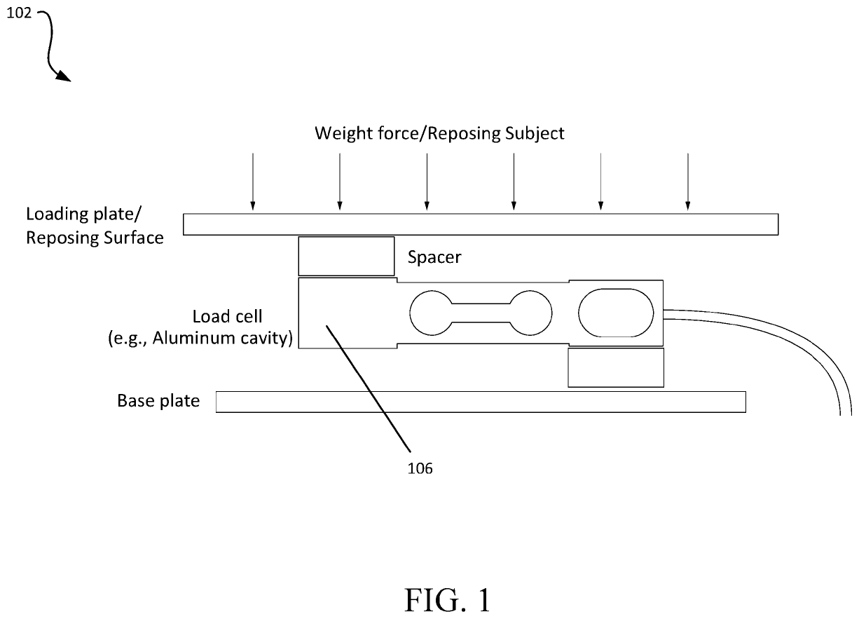

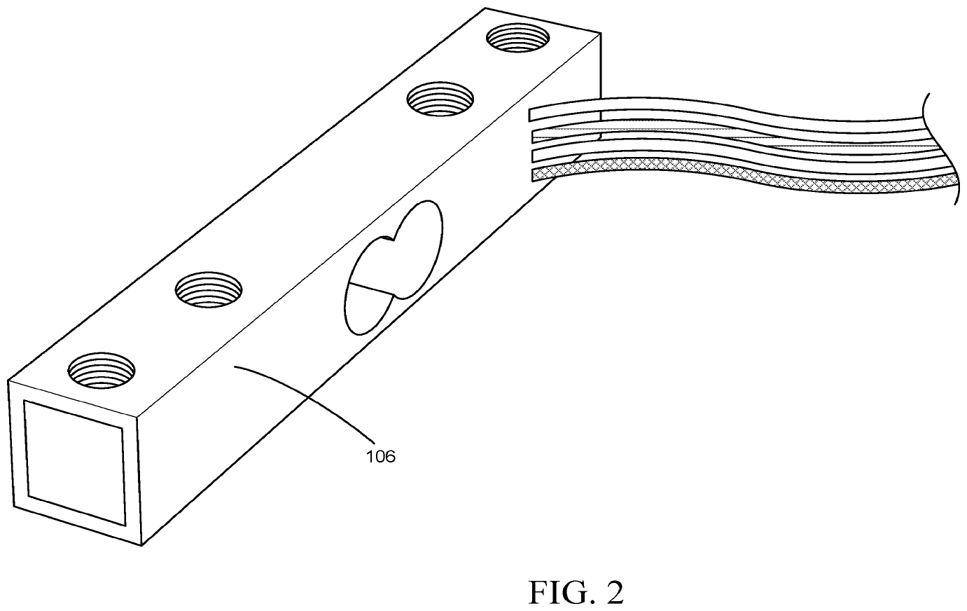

FIG. 1 illustrates an example diagram of a load cell 106 as installed with/integrated with a reposing surface 102. The load cell 106, perhaps mounted on a base plate, may translate a weight/force provided by a subject (not shown) on a reposing surface into an electrical signal for transmission to a display and/or a control device (not shown), for example. FIG. 2 illustrates a closer view of the load cell 106 of FIG. 1.

Transducers such as the load cell 106 may turn physical force into electrical signals. Load cell 106 are devices that may be utilized in measuring the weight of an object, person, dog, cat, subject, etc. Due to the epidemic and problem of subject obesity, weight is one of the most important health metrics that the subject bed will measure. Especially over time, this metric/measurement would be beyond helpful for personnel associated with the subjects and/or subjects hoping to see how healthy the subject may be. Weight may be measured through load cells, and the correct load cell choice useful in terms of weight sensitivity.

In terms of measuring the weight of an object or living animal, one component that would most likely be useful may be a printed circuit board or programmable logic controller. The purpose of this component would be to interpret the signal from the load cell 106, and output it into a readable/useful measurement.

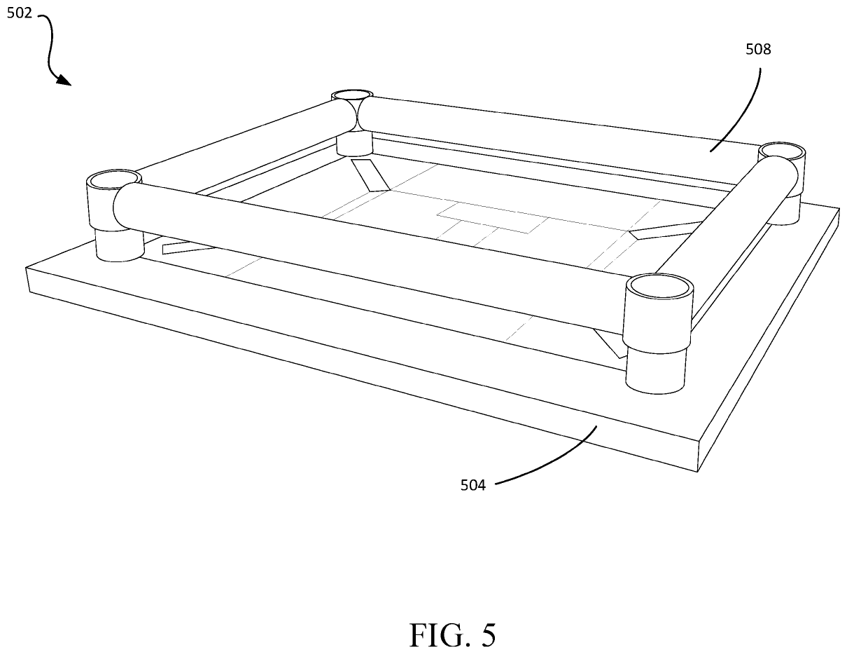

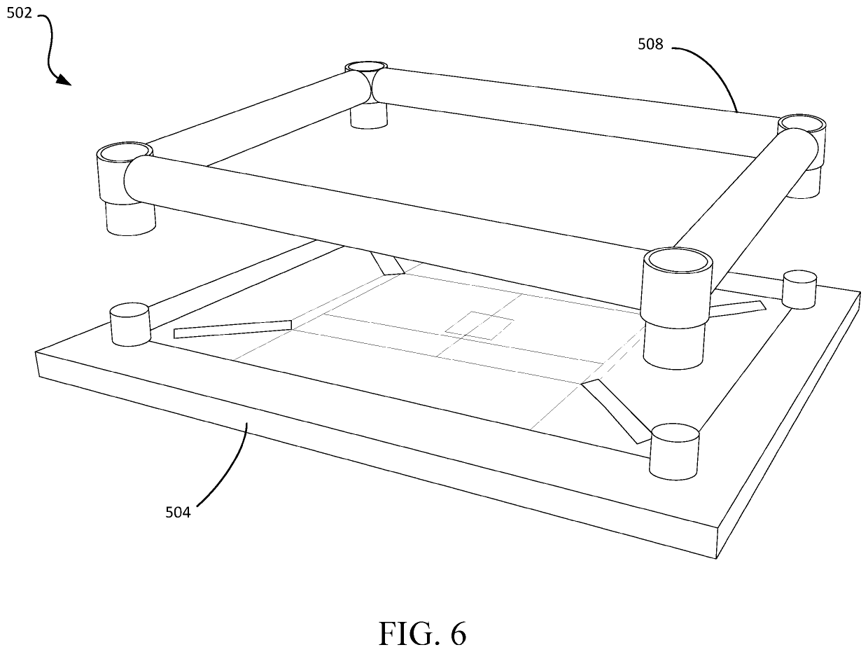

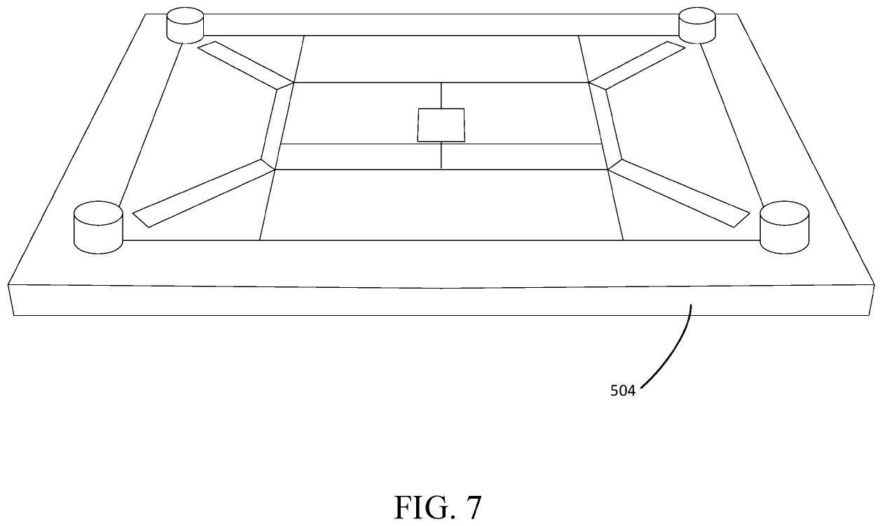

FIG. 5 illustrates a perspective view of an example of a smart bed/reposing surface base 502. In FIG. 5, a bed/resting frame 508 may be mounted on a reposing surface 504. FIG. 6 illustrates a perspective view of smart bed/reposing surface 502 where the bed/resting frame 508 is separated from the reposing surface 504. FIG. 7 illustrates a perspective view of an example of the reposing surface 504 in isolation.

Being able to obtain a body surface temperature could be useful, as this is a significantly easier to obtain alternative option that provides an idea of elevated temperature. Furthermore, subjects often hide their injuries, illnesses or otherwise compensate for poor health, making it even more difficult to evaluate. Many subjects are only infrequently seen by a doctor/veterinarian, due to factors ranging from cost to convenience, a problem which has been exacerbated by insurance, the economy, pandemics, etc.

An ongoing assessment of the subject's health through a smart bed such as the bed described herein would complement doctor/veterinarian evaluations by providing a continuous and/or objective system for data collection in between veterinary/doctor visits.

A further issue/problem that the smart bed/reposing surface may address is the lack of knowledge of subject health by typical personnel associated with the subject and/or subject. Many subjects receive infrequent health checkups by doctors/veterinarians. And during the time the doctor/veterinarian is able to observe the subject, they might not be able to observe all health issues, for example sleep apnea or seizures, or acute fever that occurs outside of a doctor/veterinarian visit.

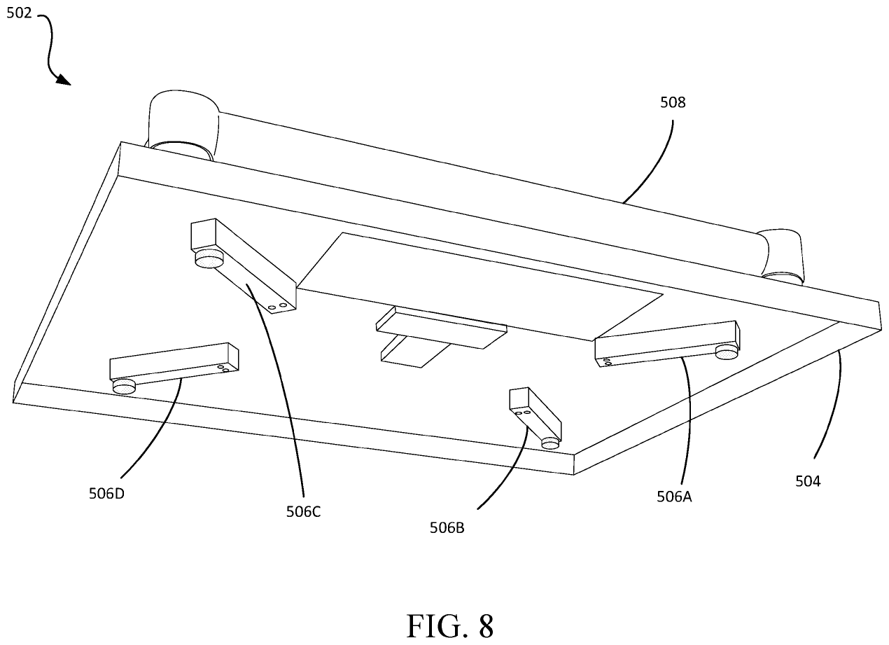

FIG. 8 illustrates an example of a perspective view of an example of a smart bed/reposing surface base 502. The bed/resting frame 508 is shown mounted on the reposing surface 504. Load cells 506A, 506B, 506C, and/or 506D may be installed on a bottom side of the reposing surface 504.

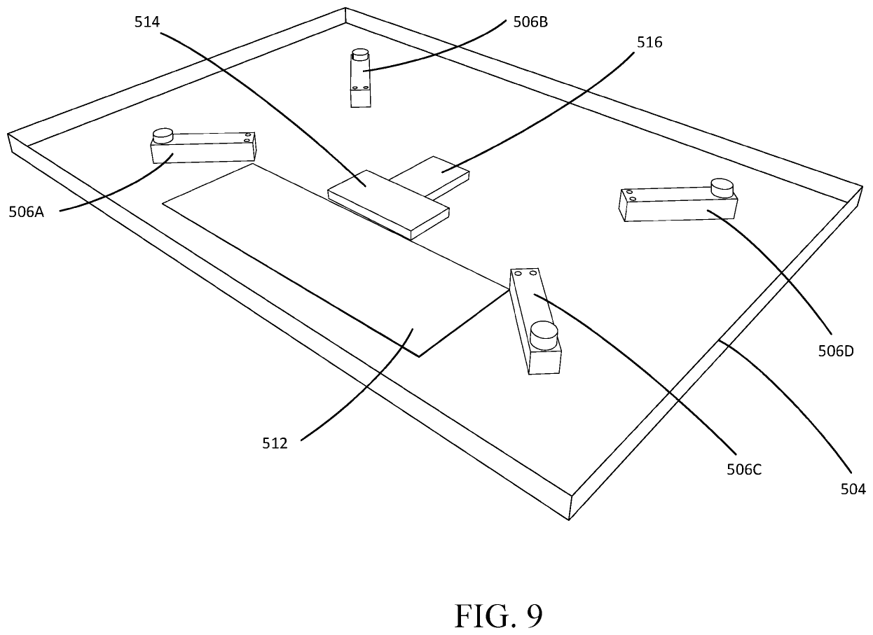

FIG. 9 illustrates an example of a perspective view of an example of the bottom side of the reposing surface 504. Load cells 506A, 506B, 506C, and/or 506D may be installed on the bottom side of the reposing surface 504. A printed circuit board 512 may be disposed on the bottom side of the reposing surface 504, among other locations. A battery cell/power source 514 may be disposed on the bottom side of the reposing surface 504, among other locations. A RFID scanning device 516 may be disposed on the bottom side of the reposing surface 504, among other locations.

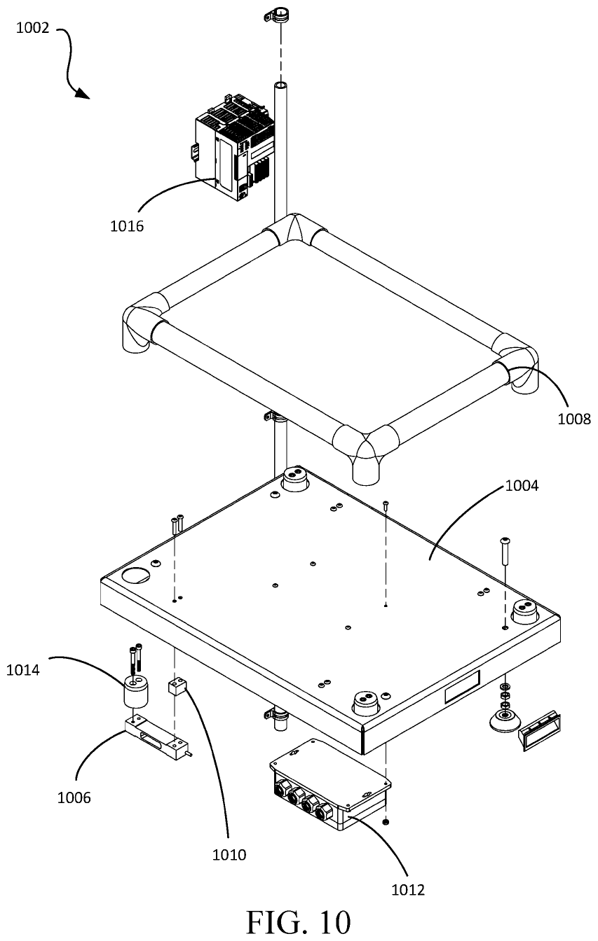

FIG. 10 illustrates an exploded view of an example of a smart bed/reposing surface base 1002. A bed/resting frame 1008 is shown that may be mountable on a reposing surface 1004. At least one load cell 1006 may be mounted to a bottom of the reposing surface 1004, perhaps for example with a mount 1014 and/or a spacer 1010. An electronic junction box/summing card assembly 1012 may be disposed on the bottom surface of the reposing surface 1004. An electronics assembly 1016 may be disposed on or near the reposing surface 1004.

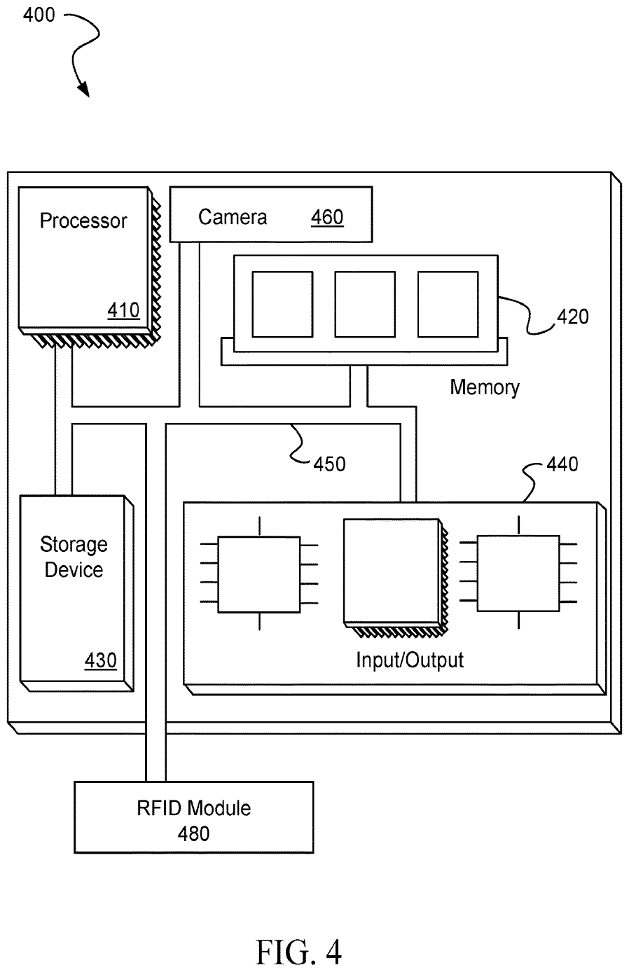

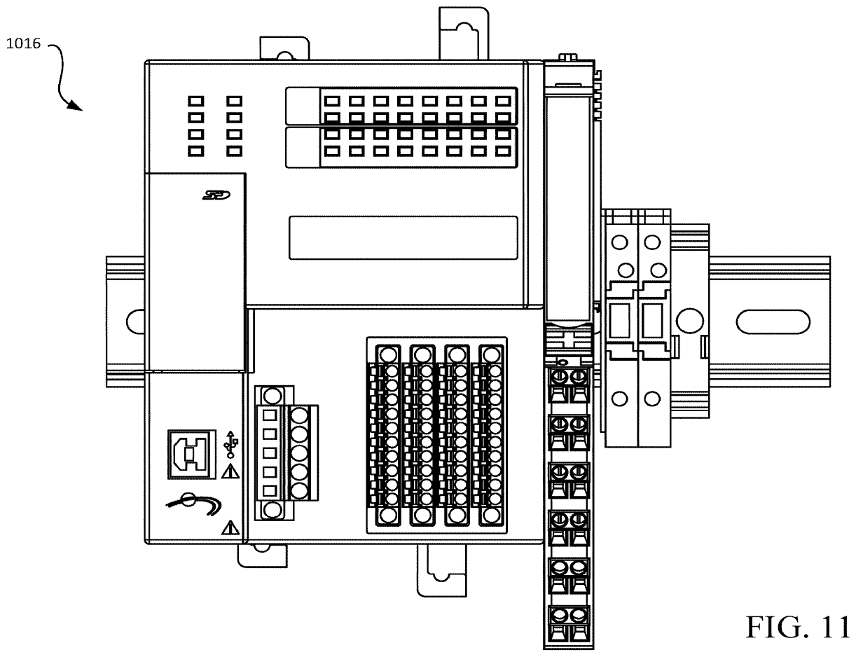

FIG. 11 is an example illustration of the electronics assembly 1016 of FIG. 10. The electronics assembly 1016 may comprise at least a part of the hardware configuration 400 of FIG. 4, for example.

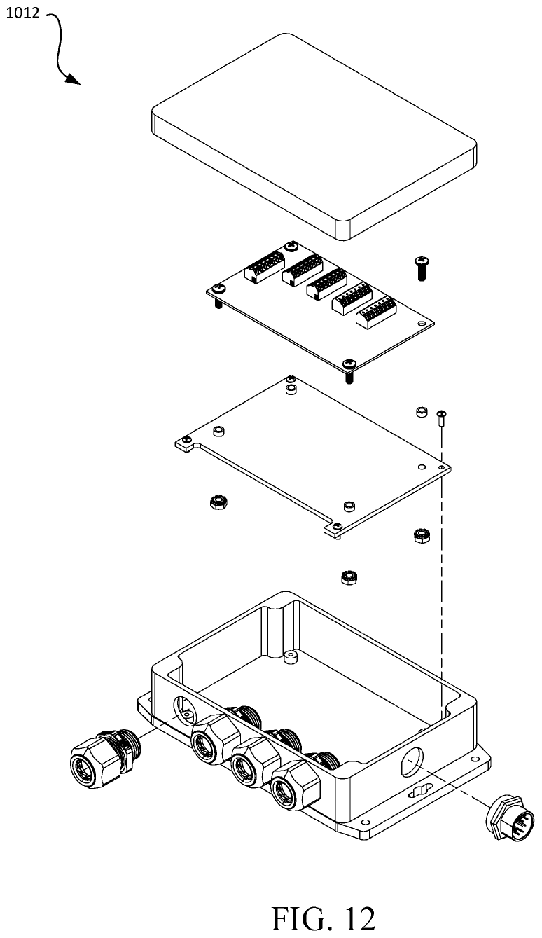

FIG. 12 is an example illustration of the electronic junction box/summing card assembly 1012 of FIG. 10. The electronic junction box/summing card assembly 1012 may be used to collect the wiring of the one or more load cells and/or other electronic components of the smart bed/reposing surface.

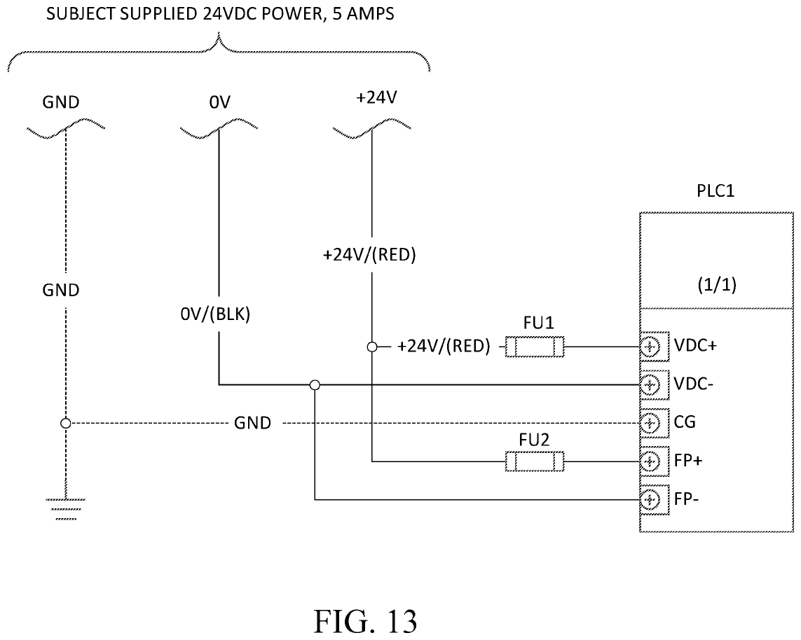

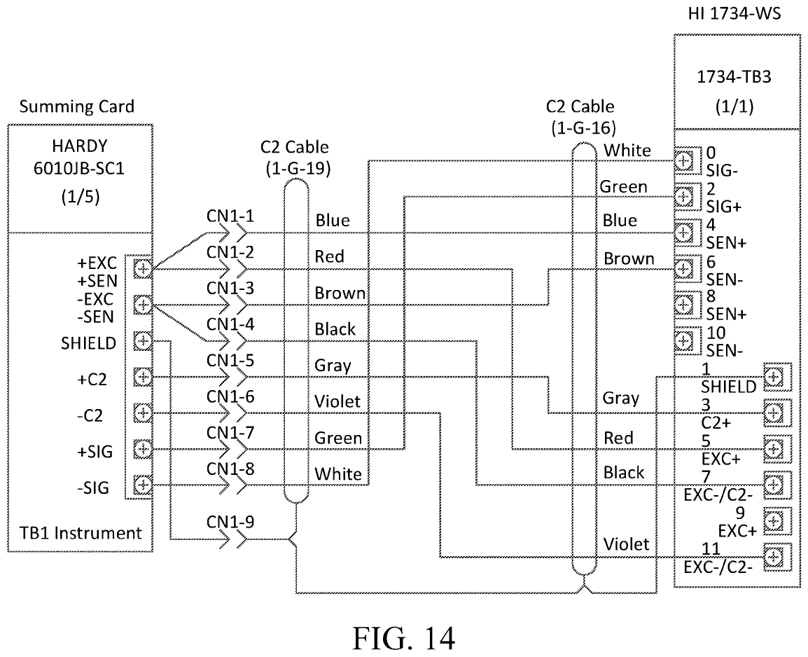

FIG. 13 is an example illustration of how, in at least one scenario, electronics and/or control devices associated with the smart bed/reposing surface may be powered for operation and/or communication. FIG. 14 is an example illustration of how, in at least one scenario, the electronic junction box/summing card assembly 1012 of FIG. 12 may be placed into electronic communication with the electronics assembly 1016 of FIG. 11.

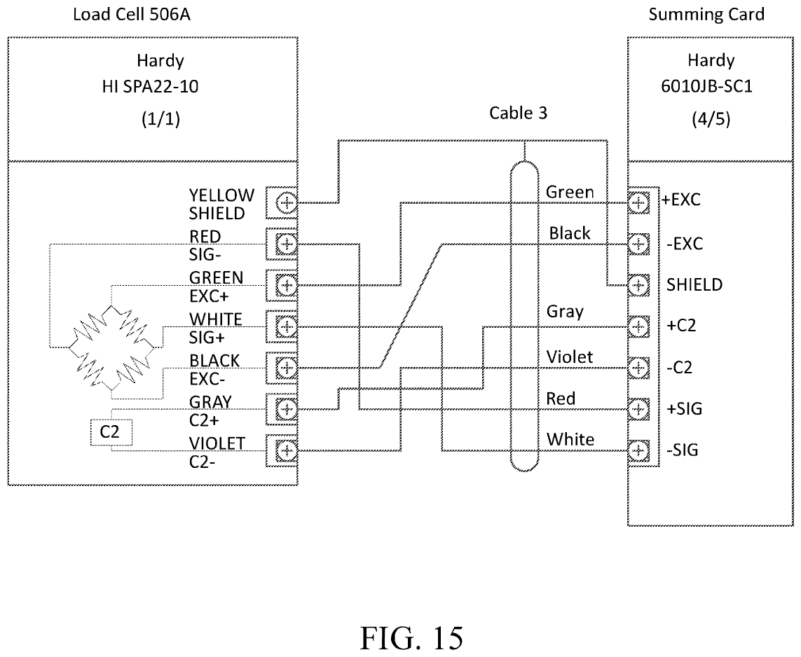

FIG. 15 is an example of how, in at least one scenario, load cell 506A of FIG. 9 may be place into electronic communication with the electronics assembly 1016 of FIG. 11, perhaps for example via the electronic junction box/summing card assembly 1012 of FIG. 12.

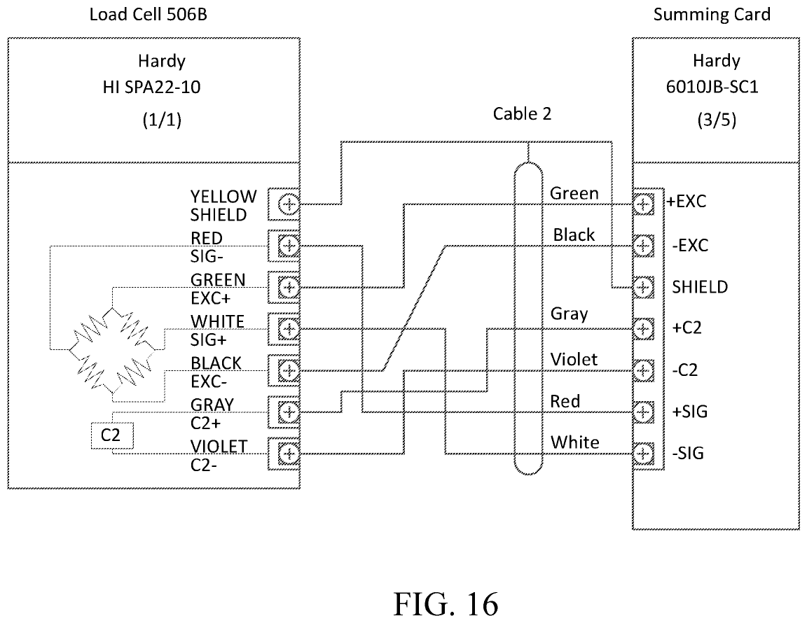

FIG. 16 is an example of how, in at least one scenario, load cell 506B of FIG. 9 may be place into electronic communication with the electronics assembly 1016 of FIG. 11, perhaps for example via the electronic junction box/summing card assembly 1012 of FIG. 12.

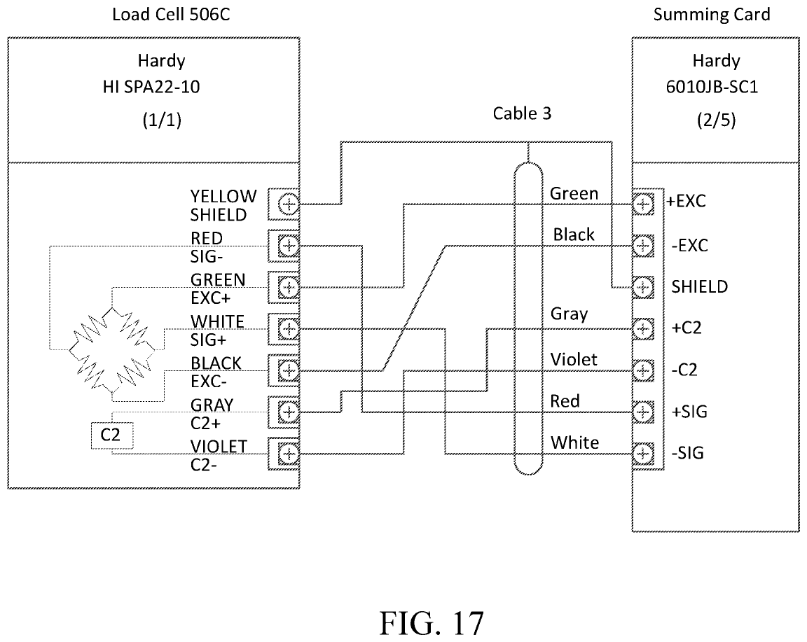

FIG. 17 is an example of how, in at least one scenario, load cell 506C of FIG. 9 may be place into electronic communication with the electronics assembly 1016 of FIG. 11, perhaps for example via the electronic junction box/summing card assembly 1012 of FIG. 12.

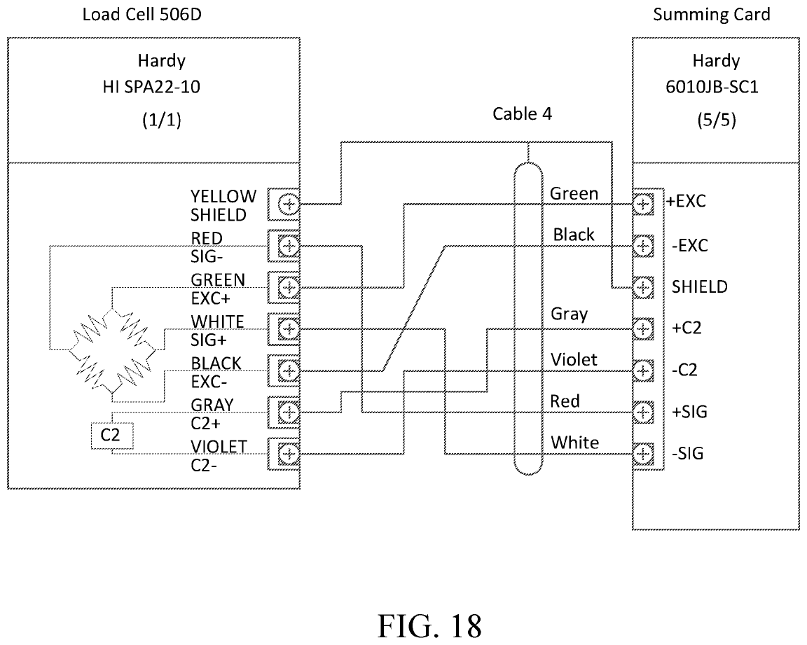

FIG. 18 is an example of how, in at least one scenario, load cell 506D of FIG. 9 may be place into electronic communication with the electronics assembly 1016 of FIG. 11, perhaps for example via the electronic junction box/summing card assembly 1012 of FIG. 12.

Significant developments in the human health space have enabled physicians or providers to obtain a better picture of what goes on with a patient's health outside the clinic. For example, companies such as Apple with the Apple Watch, or AliveCor with KardiaMobile have recently developed at home ECG monitoring systems, in order to help cardiologists understand an individual's health to a greater extent. For example, heart abnormalities can be very difficult to detect, as they can occur at random periods throughout the day. Therefore, having an additional way to detect a potentially fatal condition assists in faster diagnosis and treatment. A definite gap in the market exists for a quantification of subject health in a home environment. Therefore, with this smart bed/reposing surface, the next generation of smart veterinary/doctor technology is being developed, which could potentially assist in early detection and management of many subject health issues.

However, other features, such as those described below, may be unique to the device/data collection ecosystem described herein. The one or more algorithms used to analyze the data may be unique/specific to the device/data collection ecosystem described herein.

Proximity (e.g., integrated Bluetooth beacon that can communicate with the collar-worn/wearable to measure subject's proximity to the bed, proximity to bed/sleeping area or sleeping cubby, subject's presence in room containing bed, proximity to other subjects on or near bed, or in same room as bed, etc.).

One or more sensors could be mounted proximate to the smart bed and/or within wireless communication with the smart bed and/or a network in communication with the smart bed. One or more of the sensors may comprise: temperature sensing capabilities (e.g., body temperature, and/or ambient/environmental temperature); accelerometer for motion sensing capabilities; and/or scale/load cell capabilities which may enable monitoring of weight.

Monitoring can be conducted of the presence/absence of a subject on the bed (e.g., when does the subject arrive, depart, how long to they stay, how many times do they interact with the bed in a day, how much total time do they spend on or near the bed in a day, of the total time spent on or near the bed in a day how much is spent sleeping vs. awake, etc.)

Monitoring can be conducted of the presence/absence of multiple subjects on a bed (e.g., dog vs. cat, when does each subject arrive, depart, how long to they stay, how many times do they interact with the bed in a day, how much total time do they spend on or near the bed in a day, of the total time spent on or near the bed in a day how much is spent sleeping vs. awake, etc.).

Social interaction can be monitored/assessed, for example is the interaction of subjects on or near the bed positive or negative, etc. and/or an orientation of subject(s) on bed.

When paired with morphometric measures of the subject, body weight can be used to infer a percentage (%) of Ideal body weight, Body fat index (BFI), and body condition score (BCS).

The amount of time the subject is/is not on the bed based on weight can be used to infer total resting/sleeping time vs. non resting/sleeping time as a rough measure of calories expended and thus caloric requirements and feeding amount; Detection of growth of young animals/children over time/weight loss in older or sick animals/children; Moisture/urine/vomit/feces/blood/saliva/hairball detection (weight left on bed when subject leaves).

Devices/techniques may be paired with integrated or external video monitoring capabilities to create a smart bed system for data capture and collection which may enable monitoring of behaviors, including the following described herein. In one or more scenarios, devices/techniques may include audio monitoring capabilities, telemedicine, still images or video recordings etc., that could be paired with proximity beacons.

One or more parameters may be measured/assessed/detected, such as one or more of: Orientation of subject(s) on bed; Sleep quality (e.g., sleep disturbances such as tossing, turning vs. restful sleep), number and duration of eye movements, muscle twitches, dreaming, wake events, etc.; Breathing (e.g., respiration rate, tachypnea, bradypnea, sleep apnea, changes in respiration rate over time, and/or max/min/mean respiration rate, etc.); Seizure activity; Anxiety (e.g., shaking, whining, and/or pacing, etc. on or near bed); Moisture/urine/feces/vomit/blood/saliva visible/detectable on the bed; and/or body shape (e.g., body fat index/body condition score assessment); snoring, breathing issues, barking, whining, whimpering, chewing, purring, meowing, growling, vomiting, hairballs, urination, defecation, scratching, digging, seizures, hissing, respiratory rate, restlessness and other noises.

Paired with integrated or external audio monitoring capabilities to create a smart bed system for data capture and collection which may enable monitoring of behaviors, including the following: Snoring; Sleep apnea; Asthma; Other breathing issues (e.g., wheezing, and/or coughing, etc.); Barking; Growling; Hissing; Purring; Meowing; Whining; Vomiting; Chewing/tearing; GI function (burping, gas, bowel sounds); Breathing sounds/respiration (e.g., respiration rate, tachypnea, bradypnea, sleep apnea, changes in respiration rate over time, max/min/mean respiration rate, etc.); and/or Disturbed sleep (movement, tossing/turning, walking/pacing, restlessness, etc.); snoring, breathing issues, barking, whining, whimpering, chewing, purring, meowing, growling, vomiting, hairballs, urination, defecation, scratching, digging, seizures, hissing, respiratory rate, restlessness and other noises.

Other functionality may include sensors for detection of one or more of the following: Heart Rate (e.g., tachycardia, bradycardia, and/or asystole); seizures; and/or body outline/BFI or BCS scanner, for example.

In one or more scenarios, on-device (integrated) or remote (server/cloud) machine learning algorithms for data analysis including behavior detection, trend monitoring and automated alert generation. Examples of information reported/processed by one or more algorithms may include alerts for weight gain/loss over time, respiratory alert (e.g., breathing too slow, too fast, and/or absence of breathing), presence/absence on or near bed alert, heart rate alert (e.g., heart rate too slow, too fast, and/or absence of heart rate), body temperature and/or ambient temperature alert (e.g., too warm, or too cold), and/or seizure alert, etc.

Other features may include heating, cooling, vibration alarm to wake subject, audio alarm to wake subject, sleep apnea or dreaming alarm to wake subject from sleep apnea or dreaming, owner voice integration to soothe an anxious subject or help the subject fall asleep, treat dispenser, rocking motion to help subject fall asleep, calming scents to soothe anxious subject or help subject fall asleep, removable cover, etc.

As referenced herein, the phrase “smart bed” may refer to a bed/reposing surface that focuses on collection of data related to the health and wellbeing of the subject. It may be suitable for humans and/or animals (e.g., pets). It contains electronics/power source, may records and/or store data. It may or might not interact with other devices, may or might not pass data to programs for analysis. One or more features may include the following:

One or more devices and/or methods described herein may collect and/or save data from a variety of sensors and/or data capture technologies through an internal memory chip. One or more devices and/or methods/techniques described herein may transfer data from the smart bed/network using a PLC, Wi-Fi, and/or BLE interface, among other technologies.

One or more devices and/or methods described herein may contain electronics and/or may be powered by PLC or external power supply. One or more devices and/or methods described herein may collect data into an internal and/or external database to be queried and/or referenced by one or more, or numerous, programs.

One or more devices and/or methods described herein may or might not interface with other devices, such as RFID, to provide additional information at time of scanning such as subject identification, subject temperature, and/or wearable data, etc.

One or more devices and/or methods described herein may be configured to detect subject body weight changes over time.

One or more devices/techniques may be paired with integrated or external video and/or audio monitoring capabilities, and/or telemedicine to create a smart bed system for data capture and collection which may enable monitoring of behaviors, including one or more of the technologies and/or techniques disclosed herein.

One or more devices disclosed herein, and/or techniques performed by the one or more devices, may include use/detection/assessment of one or more of: RFID; Seizure activity; Anxiety (e.g., shaking, whining, pacing, etc. on or near bed); Moisture/urine/feces/vomit/blood/saliva visible/detectable on the bed; body shape (e.g., body fat index/body condition score assessment); barking; growling; hissing; purring; meowing; whining; vomiting; chewing/tearing; GI function (e.g., burping, gas, and/or bowel sounds); and/or social interaction, such as for example—is the interaction of subjects on or near the bed positive or negative, etc. Snoring; Sleep apnea; Asthma; Other breathing issues (e.g., wheezing, and/or coughing, etc.); Barking; Growling; Hissing; Purring; Meowing; Whining; Vomiting; Chewing/tearing; GI function (burping, gas, bowel sounds); Breathing sounds/respiration (e.g., respiration rate, tachypnea, bradypnea, sleep apnea, changes in respiration rate over time, max/min/mean respiration rate, etc.); and/or Disturbed sleep (movement, tossing/turning, walking/pacing, restlessness, etc.); snoring, breathing issues, barking, whining, whimpering, chewing, purring, meowing, growling, vomiting, hairballs, urination, defecation, scratching, digging, seizures, hissing, respiratory rate, restlessness and other noises.

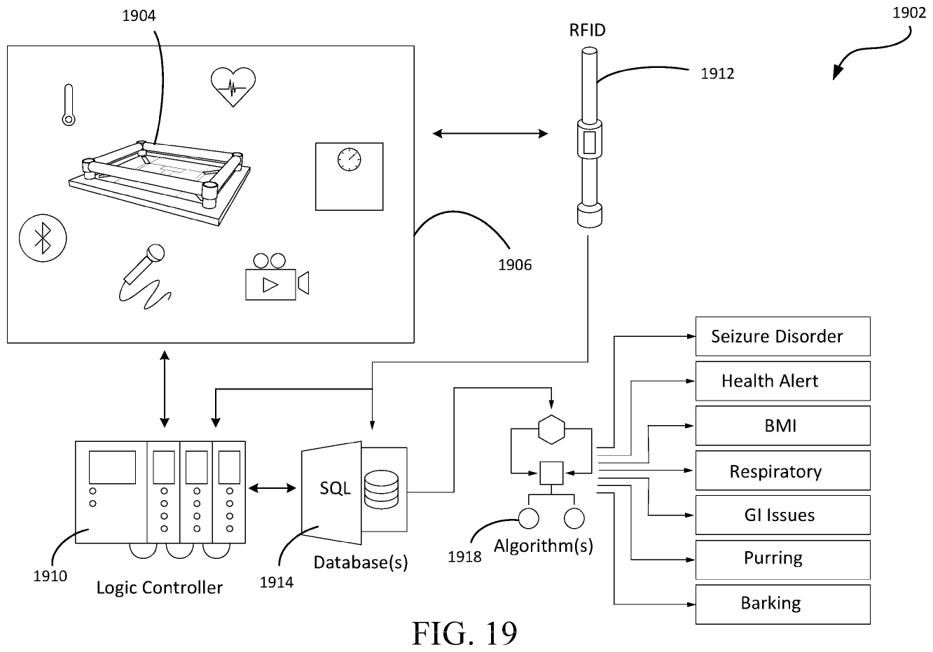

FIG. 19 is an example diagram of a health parameter collection network 1902 that includes a smart bed 1904. The smart bed 1904 may comprise a group of one or more sensors 1906, and/or may be in communication with the group of one or more sensors 1906. The group of one or more sensors 1906 may comprise a heart monitor, a microphone, a camera, a temperature sensor, and/or a weight scale. The smart bed 1904 may have one or more load cells installed under its base (not shown). The smart bed 1904 may be in communication with a number of other sensors (not shown) via a Bluetooth connection and/or via another communication protocol.

The smart bed 1904 and/or the group of one or more sensors 1906 may be in communication with an RFID scanning device/antenna 1912. The RFID scanning device 1912 may be in communication with a logic controller 1910 and/or one or more databases 1914. The logic controller 1910 may comprise one or more of the components of the hardware configuration 400 of FIG. 4, for example.

The logic controller 1910 may receive one or more signals from any of the smart bed 1904, the RFID scanner device 1912, and/or any of the sensors of the group of one or more sensors 1906. The logic controller 1906 may be configured to receive one or more signals from any of the devices of the health parameter collection network 1902. The logic controller 1904 may be configured to interpret any of the signals as characteristics corresponding to a subject (not shown) that may occupy the environment in which the health parameter collection network 1902 may operate/monitor.

The logic controller 1910 may be configured with, and/or may be in communication with, the one or more databases 1914 and/or one or more algorithms 1918. The logic controller 1904 may be configured (e.g., using the one or more databases 1914 and/or the one or more algorithms 1918) to determine one or more indications of the subject's health based, at least in part, on the one or more characteristics corresponding to the subject.

The one or more indications of the subject's health may comprise an indication of a seizure disorder, a health alert (e.g., general or specific), a body-mass index (BMI) indication, a respiratory indication, a gastrointestinal (GI) issue, a purring activity (e.g., a pet cat being the subject), and/or a barking activity (e.g., a pct dog being the subject), among other indications, for example.

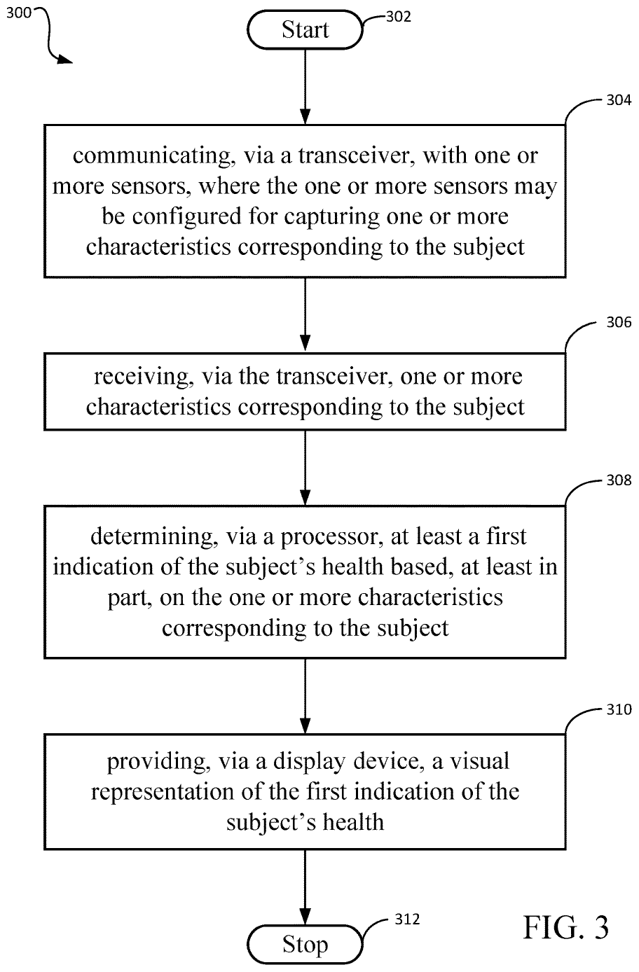

Referring now to FIG. 3, a diagram 300 illustrates an example method/technique/process for assessing a subject's health based on health-related information. The method may be performed by a logic controller/processor, among other devices. For example, the hardware configuration 400 of FIG. 4, the Logic Controller of FIG. 10, and/or a cloud computing device. The logic controller/processor may be in communication with one or more sensors and/or a display device. At 302, the process may start or restart.

At 304, the process may comprise communicating, for example via a transceiver, with the one or more sensors. The one or more sensors may be configured for capturing one or more characteristics corresponding to the subject. At 306, the process may comprise receiving, for example via the transceiver, one or more characteristics corresponding to the subject.

At 308, the process may comprise determining, for example via a processor, at least a first indication of the subject's health based, at least in part, on the one or more characteristics corresponding to the subject.

At 310, the process may comprise providing, for example via the display device, a visual representation of the first indication of the subject's health. At 312 the process may stop or restart.

FIG. 4 is a block diagram of a hardware configuration of an example device that may function as a process control device/logic controller, for example. The hardware configuration 400 may be operable to facilitate delivery of information from an internal server of a device. The hardware configuration 400 can include a processor 410, a memory 420, a storage device 430, and/or an input/output device 440. One or more of the components 410, 420, 430, and 440 can, for example, be interconnected using a system bus 450. The processor 410 can process instructions for execution within the hardware configuration 400. The processor 410 can be a single-threaded processor or the processor 410 can be a multi-threaded processor. The processor 410 can be capable of processing instructions stored in the memory 420 and/or on the storage device 430.

The memory 420 can store information within the hardware configuration 400. The memory 420 can be a computer-readable medium (CRM), for example, a non-transitory CRM. The memory 420 can be a volatile memory unit, and/or can be a non-volatile memory unit.

The storage device 430 can be capable of providing mass storage for the hardware configuration 400. The storage device 430 can be a computer-readable medium (CRM), for example, a non-transitory CRM. The storage device 430 can, for example, include a hard disk device, an optical disk device, flash memory and/or some other large capacity storage device. The storage device 430 can be a device external to the hardware configuration 400.

The input/output device 440 may provide input/output operations for the hardware configuration 400. The input/output device 440 (e.g., a transceiver device) can include one or more of a network interface device (e.g., an Ethernet card), a serial communication device (e.g., an RS-232 port), one or more universal serial bus (USB) interfaces (e.g., a USB 2.0 port) and/or a wireless interface device (e.g., an 802.11 card). The input/output device can include driver devices configured to send communications to, and/or receive communications from one or more networks. The input/output device 400 may be in communication with one or more input/output modules (not shown) that may be proximate to the hardware configuration 400 and/or may be remote from the hardware configuration 400. The one or more output modules may provide input/output functionality in the digital signal form, discrete signal form, TTL form, analog signal form, serial communication protocol, fieldbus protocol communication and/or other open or proprietary communication protocol, and/or the like.

A Radio Frequency Identification (RFID) Module 480 may scan/receive various forms of RFID communication. In one or more scenarios, the RFID Module 480 may be mounted on/proximate to the hardware configuration 400. In one or more scenarios, the RFID Module 480 may be remote from the hardware configuration 400. The RFID module 480 may be in wired and/or wireless communication with the hardware configuration 400 and/or any of the components of the hardware configuration 400.

The camera device 460 may provide digital video input/output and audio input/output (e.g., for telemedicine) capability for the hardware configuration 400. The camera device 460 may communicate with any of the elements of the hardware configuration 400, perhaps for example via system bus 450. The camera device 460 may capture digital images and/or may scan images of various kinds, such as Universal Product Code (UPC) codes (e.g., bar codes) and/or Quick Response (QR) codes (e.g., two-dimensional bar codes), for example, among other images as described herein. In one or more scenarios, the camera device 460 may be the same and/or substantially similar to any of the other camera devices described herein.

The camera device 460 may include at least one microphone device and/or at least one speaker device. The input/output of the camera device 460 may include audio signals/packets/components, perhaps for example separate/separable from, or in some (e.g., separable) combination with, the video signals/packets/components the camera device 460.

The camera device 460 may also detect the presence of one or more people/subjects/pets that may be proximate to the camera device 460 and/or may be in the same general space (e.g., the same room) as the camera device 460. The camera device 460 may gauge a general activity level (e.g., high activity, medium activity, and/or low activity) of one or more people/subjects/pets that may be detected by the camera device 460. The camera device 460 may detect one or more general characteristics (e.g., height, body shape, skin color, pulse, heart rate, breathing count, etc.) of the one or more people/subjects/pets detected by the camera device 460. The camera device 460 may be configured to recognize one or more specific people/subject/pets, for example.

The camera device 460 may be in wired and/or wireless communication with the hardware configuration 400. In one or more scenarios, the camera device 460 may be external to the hardware configuration 400. In one or more scenarios, the camera device 460 may be internal to the hardware configuration 400.

The subject matter of this disclosure, and components thereof, can be realized by instructions that upon execution cause one or more processing devices to carry out the processes and/or functions described herein. Such instructions can, for example, comprise interpreted instructions, such as script instructions, e.g., JavaScript or ECMAScript instructions, or executable code, and/or other instructions stored in a computer readable medium.

Implementations of the subject matter and/or the functional operations described in this specification and/or the accompanying figures can be provided in digital electronic circuitry, in computer software, firmware, and/or hardware, including the structures disclosed in this specification and their structural equivalents, and/or in combinations of one or more of them. The subject matter described in this specification can be implemented as one or more computer program products, e.g., one or more modules of computer program instructions encoded on a tangible program carrier for execution by, and/or to control the operation of, data processing apparatus.

A computer program (also known as a program, software, software application, script, or code) can be written in any form of programming language, including compiled or interpreted languages, and/or declarative or procedural languages. It can be deployed in any form, including as a stand-alone program or as a module, component, subroutine, and/or other unit suitable for use in a computing environment. A computer program may or might not correspond to a file in a file system. A program can be stored in a portion of a file that holds other programs and/or data (e.g., one or more scripts stored in a markup language document), in a single file dedicated to the program in question, and/or in multiple coordinated files (e.g., files that store one or more modules, sub programs, or portions of code). A computer program can be deployed to be executed on one computer or on multiple computers that may be located at one site or distributed across multiple sites and/or interconnected by a communication network.

The processes and/or logic flows described in this specification and/or in the accompanying figures may be performed by one or more programmable processors executing one or more computer programs to perform functions by operating on input data and/or generating output, thereby tying the process to a particular machine (e.g., a machine programmed to perform the processes described herein). The processes and/or logic flows can also be performed by, and apparatus can also be implemented as, special purpose logic circuitry, e.g., an FPGA (field programmable gate array) and/or an ASIC (application specific integrated circuit).

Computer readable media suitable for storing computer program instructions and/or data may include all forms of non-volatile memory, media and memory devices, including by way of example semiconductor memory devices (e.g., EPROM, EEPROM, and/or flash memory devices); magnetic disks (e.g., internal hard disks or removable disks); magneto optical disks; and/or CD ROM and DVD ROM disks. The processor and/or the memory can be supplemented by, or incorporated in, special purpose logic circuitry.

While this specification and the accompanying figures contain many specific implementation details, these should not be construed as limitations on the scope of any invention and/or of what may be claimed, but rather as descriptions of features that may be specific to described example implementations. Certain features that are described in this specification in the context of separate implementations can also be implemented in combination in perhaps one implementation. Various features that are described in the context of perhaps one implementation can also be implemented in multiple combinations separately or in any suitable sub-combination. Although features may be described above as acting in certain combinations and/or perhaps even (e.g., initially) claimed as such, one or more features from a claimed combination can in some cases be excised from the combination. The claimed combination may be directed to a sub-combination and/or variation of a sub-combination.

While operations may be depicted in the drawings in an order, this should not be understood as requiring that such operations be performed in the particular order shown and/or in sequential order, and/or that all illustrated operations be performed, to achieve useful outcomes. The described program components and/or systems can generally be integrated together in a single software product and/or packaged into multiple software products.

Examples of the subject matter described in this specification have been described. The actions recited in the claims can be performed in a different order and still achieve useful outcomes, unless expressly noted otherwise. For example, the processes depicted in the accompanying figures do not require the particular order shown, and/or sequential order, to achieve useful outcomes. Multitasking and parallel processing may be advantageous in one or more scenarios.

While the present disclosure has been illustrated and described in detail in the drawings and foregoing description, the same is to be considered as illustrative and not restrictive in character, it being understood that only certain examples have been shown and described, and that all changes and modifications that come within the spirit of the present disclosure are desired to be protected.

1. A system for assessing a subject's health based on health-related information, the system comprising:
a memory;
a display device;
a transceiver, the transceiver being in communication with one or more sensors, the one or more sensors configured to capture one or more characteristics corresponding to the subject; and
a processor, the processor configured at least to:
receive one or more characteristics corresponding to the subject via the transceiver;
determine at least a first indication of the subject's health based, at least in part, on the one or more characteristics corresponding to the subject; and
provide a visual representation of the first indication of the subject's health via the display device.

2. The system of claim 1, further comprising at least one reposing surface on which the subject reposes for at least some time on a daily basis.

3. The system of claim 1, wherein at least one of the one or more sensors are disposed in at least one of: physical contact with the reposing surface, proximate to the reposing surface, or within a line of sight of the reposing surface.

4. The system of claim 1, wherein the transceiver is configured to communicate with the one or more sensors via at least one of: wired communication, or wireless communication.

5. (canceled)

6. The system of claim 1, wherein the subject is at least one of: an animal, or a human, and at least one of the one or more sensors is a load cell.

7. The system of claim 1, wherein the one or more characteristics corresponding to the subject comprise: a physical parameter corresponding to the subject, an image corresponding to the subject, an identification corresponding to the subject, a sound corresponding to the subject, an activity corresponding to the subject, a behavior corresponding to the subject, a motion corresponding to the subject, a location corresponding to the subject, and a manually input observation corresponding to the subject.

8. The system of claim 7, wherein the physical parameter corresponding to the subject comprises: a temperature corresponding to the subject, a weight corresponding to the subject, a breathing rate corresponding to the subject, or a heart rate corresponding to the subject, moisture detectable on a reposing surface corresponding to the subject, urine detectable on the reposing surface corresponding to the subject, feces detectable on the reposing surface corresponding to the subject, vomit detectable on a reposing surface corresponding to the subject, blood detectable on a reposing surface corresponding to the subject, saliva detectable on a reposing surface corresponding to the subject, a body shape corresponding to the subject, a body fat index corresponding to the subject, and a body condition score assessment corresponding to the subject.

9. The system of claim 7, wherein the image corresponding to the subject comprises one of more of: a video recording corresponding to the subject, or one or more still image frames corresponding to the subject.

10. The system of claim 7, wherein the sound corresponding to the subject comprises one or more of: a gastro-intestinal sound corresponding to the subject, a vocal sound corresponding to the subject, a pulmonary sound corresponding to the subject, respiratory sound emission corresponding to the subject, or environmental sound corresponding to the subject, a chewing sound corresponding to the subject, a tearing sound corresponding to the subject, a growling sound corresponding to the subject, a purring sound corresponding to the subject, a meowing sound corresponding to the subject, a hissing sound corresponding to the subject, a whining sound corresponding to the subject, a whimpering sound corresponding to the subject, a hairball-related sound corresponding to the subject, a vomiting sound corresponding to the subject barking, a burping sound corresponding to the subject, a gas sound corresponding to the subject, a bowel sound corresponding to the subject, or a social interaction-based sound corresponding to the subject.

11. The system of claim 7, wherein the activity corresponding to the subject comprises one of more of: a social interaction corresponding to the subject, a sleeping event corresponding to the subject, a resting event corresponding to the subject, an exercise event corresponding to the subject, or medical event corresponding to the subject.

12. The system of claim 7, wherein the behavior corresponding to the subject comprises one or more of: an indication of anxiety corresponding to the subject, a demonstration of anger corresponding to the subject, an indication of happiness corresponding to the subject, an indication of apprehension corresponding to the subject, or an indication of illness corresponding to the subject.

13. The system of claim 7, wherein the motion corresponding to the subject comprises one or more of: a scratching motion corresponding to the subject, a heavy-breathing motion corresponding to the subject, a limping motion corresponding to the subject, a running motion corresponding to the subject, or a jumping motion corresponding to the subject.

14. The system of claim 7, wherein the identification corresponding to the subject comprises one or more of: a radio frequency identification (RFID) corresponding to the subject, a barcode identification corresponding to the subject, or a two-dimensional barcode corresponding to the subject, an identification based on one or more physiological measurements corresponding to the subject, an identification based on a wearable device, an identification based on an implanted device, an identification based on an injected device, an identification based on one or more audio signals corresponding to the subject, an identification based on one or more images corresponding to the subject, an identification based on an identity of the reposing surface, or an identification based on at least one of: a weight corresponding to the subject, a temperature corresponding to the subject, a heart rate corresponding to the subject, a respiration rate corresponding to the subject, a pattern of movement corresponding to the subject, or a time of day.

15. The system of claim 7, wherein the manually input observation corresponding to the subject comprises one or more of: a medical diagnosis corresponding to the subject, an observation provided via a telemedicine session, a physical therapist evaluation corresponding to the subject, an untrained-observer observation corresponding to the subject, a dietary record corresponding to the subject, or a medication record corresponding to the subject.

16. The system of claim 7, wherein the location corresponding to the subject comprises one or more of: a geography corresponding to the subject, a region corresponding to the subject, global positioning system (GPS) coordinates corresponding to the subject, a housing area corresponding to the subject, a room of a housing area corresponding to the subject, a region of a housing area corresponding to the subject, a determination of at least one of: an indoor location corresponding to the subject or an outdoor location corresponding to the subject, or a primary residential neighborhood corresponding to the subject.

17. The system of claim 1, wherein to determine the at least first indication of the subject's health, the processor is further configured to:
select at least one of the one or more characteristics corresponding to the subject;
process the at least one characteristic through one or more algorithms corresponding to the at least first indication of the subject's health; and
determine the at least first indication of the subject's heath based, at least in part, on the one or more algorithms.

18. The system of claim 1, wherein the processor is configured such that the first indication of the subject's health is at least one of: a weight gain over time, a weight loss over time, a gastro-intestinal change over time, a behavioral change over time, a seizure behavior change over time, an anxiety behavior change over time, an aggressive behavior change over time, a respiratory alert, a previously diagnosed mental state change over time, a vomiting behavior change over time, a urination frequency change over time, a defecation frequency change over time, a hairball behavior change over time, a presence proximate to the reposing surface indication, an absence proximate to the reposing surface, a heart rate alert, a body temperature alert, an ambient temperature alert, a seizure alert, a sleep quality change over time, a breathing rate change over time, a heart rate change over time, a movement pattern change over time, a social interaction change over time, a snoring change over time, a sleep apnea condition, an asthma condition, a wheezing condition, a coughing condition, a barking behavior, a growling behavior, a hissing behavior, a purring behavior, a meowing behavior, a whining behavior, a vomiting behavior, a chewing behavior, a tearing behavior, a burping behavior, a bowel condition, a respiration rate change over time, a disturbed sleep condition, or a restlessness condition.

19. The system of claim 18, wherein any of the changes comprise a change in: a presence, an absence, a frequency, an occurrence, an amount, and a severity.

20. (canceled)

21. The system of claim 1, wherein the one or more sensors are at least one of: a wearable sensor, a remote sensor, a user-input sensor, a barcode sensor, an implanted sensor, an injected sensor, a radio frequency identification (RFID) sensor.

22. A method for assessing a subject's health based on health-related information, the method comprising:
communicating, via a transceiver, with one or more sensors, the one or more sensors configured for capturing one or more characteristics corresponding to the subject;
receiving, via the transceiver, one or more characteristics corresponding to the subject;
determining, via a processor, at least a first indication of the subject's health based, at least in part, on the one or more characteristics corresponding to the subject; and
providing, via a display device, a visual representation of the first indication of the subject's health.

23. The method of claim 22, wherein the determining the at least first indication of the subject's health further comprises:
selecting at least one of the one or more characteristics corresponding to the subject;
processing the at least one characteristic through one or more algorithms corresponding to the at least first indication of the subject's health; and
determining the at least first indication of the subject's heath based, at least in part, on the one or more algorithms;
wherein the subject is at least one of: an animal, or a human, and at least one of the one or more sensors is a load cell.

24. (canceled)

25. The method of claim 22, wherein the one or more characteristics corresponding to the subject comprise a physical parameter corresponding to the subject, an image corresponding to the subject, an identification corresponding to the subject, a sound corresponding to the subject, an activity corresponding to the subject, a behavior corresponding to the subject, a motion corresponding to the subject, a location corresponding to the subject, or a manually input observation corresponding to the subject.

26. The method of claim 22, wherein at least one of the one or more sensors are disposed in at least one of: physical contact with a reposing surface, proximate to the reposing surface, or within a line of sight of the reposing surface, the reposing surface being a surface on which the subject reposes for at least some time on a daily basis.

27. (canceled)

28. The system of claim 1, further comprising one or both of a heating element and a cooling element.

29. The method of claim 22, further comprising the step of applying one or both of a heating and a cooling treatment to the subject.
